(19) United States
(12) Patent Application Publication
(10) Pub. No.: US 2025/0281221 A1
(43) Pub. Date: Sep. 11, 2025

(54) INTRAVASCULAR LITHOTRIPSY CATHETER SYSTEM WITH CONTROLLER AND GRAPHICAL USER INTERFACE

(71) Applicant: BOLT MEDICAL, INC., Carlsbad, CA (US)

(72) Inventors: Christopher A. Cook, Laguna Niguel, CA (US); Eric Schultheis, San Clemente, CA (US); Rachel Troutman, Carlsbad, CA (US); Lakshmi Narayan Mishra, Carlsbad, CA (US)

(21) Appl. No.: 19/068,673

(22) Filed: Mar. 3, 2025

Related U.S. Application Data
(60) Provisional application No. 63/561,448, filed on Mar. 5, 2024.

(51) Int. Cl. A61B 18/04; A61B 17/22; A61B 18/00

(52) U.S. Cl. CPC A61B 18/042; A61B 2017/22067; A61B 2018/0022; A61B 2018/00369

(57) ABSTRACT

A catheter system (100) usable for treating a treatment site (106) within or adjacent to a vessel wall (108A) of a blood vessel (108) or a heart valve within a body (107) of a patient (109), the catheter system (100) can include an energy source (124) that generates energy, a catheter (102) that includes an energy guide (122A) that receives the energy from the energy source (124), and a system controller (126) that is coupled to the energy source (124), the system controller (126) being configured to: (i) monitor real-time operating parameters of the catheter system (100), including at least energy delivery characteristics and treatment site conditions, (ii) detect and identify operating parameters that require adjustment, (iii) automatically adjust operating parameters, and (iv) dynamically provide a summary of system status, treatment progress, and actionable alerts.

### RELATED APPLICATION

This Application claims priority on U.S. Provisional Patent Application Ser. No. 63/561,448 filed on Mar. 5, 2024, and entitled “GRAPHICAL USER INTERFACE FOR INTRAVASCULAR LITHOTRIPSY CATHETER SYSTEM”. To the extent permitted, the contents of U.S. Provisional Patent Application Ser. No. 63/561,448 are incorporated in their entirety herein by reference.

### BACKGROUND

Vascular lesions (also referred to herein as a “treatment site”) within and/or adjacent to vessels in the body can be associated with an increased risk for major adverse events, such as myocardial infarction, embolism, deep vein thrombosis, stroke, and the like. Severe vascular lesions, such as severely calcified vascular lesions, can be difficult to treat and achieve patency for a physician in a clinical setting.

Vascular lesions may be treated using interventions such as drug therapy, balloon angioplasty, atherectomy, stent placement, and vascular graft bypass, to name a few. Such interventions may not always be ideal or may require subsequent treatment to address the lesion.

Intravascular lithotripsy is one method that has been recently used with some success for breaking up vascular lesions within vessels in the body. Intravascular lithotripsy utilizes a combination of pressure waves and bubble dynamics that are generated intravascularly in a fluid-filled balloon catheter. In particular, in certain implementations of an intravascular lithotripsy treatment, a high energy source is used to provide energy to an energy guide and/or an emitter in order to generate plasma and, ultimately, pressure waves as well as a rapid bubble expansion within a fluid-filled balloon to crack calcification at a treatment site within the vasculature that includes one or more vascular lesions. The associated rapid bubble formation from the plasma initiation and resulting localized fluid velocity within the balloon transfers mechanical energy through the incompressible fluid to impart a fracture force on the intravascular calcium, which is opposed to the balloon wall. The rapid change in fluid momentum upon hitting the balloon wall is known as hydraulic shock or water hammer.

Conventional intravascular lithotripsy catheter systems include user interfaces that provide minimal information to the user about the catheter system. In some instances, the user interfaces of these catheter systems only indicate the number of pulses remaining on the catheter and the balloon size of the connected device. Due to the lack of information provided by the user interface, clinicians have to rely on other external devices such as endoflator pressure gauges and stopwatches or timers to get other relevant information during an intravascular lithotripsy procedure.

### SUMMARY

The present invention is directed toward a catheter system usable for treating a treatment site within or adjacent to a vessel wall of a blood vessel or a heart valve within a body of a patient. The catheter system includes an energy source that generates energy, a catheter, and a system controller. The system controller is coupled to the energy source. The system controller being configured to: (i) monitor real-time operating parameters of the catheter system, including at least energy delivery characteristics and treatment site conditions, (ii) detect and identify operating parameters that require adjustment, (iii) automatically adjust operating parameters, and (iv) dynamically provide a summary of system status, treatment progress, and actionable alerts.

In some embodiments, the catheter system further comprises a communicator that is coupled to the system controller, the communicator being configured to communicate treatment feedback and system status updates.

In certain embodiments, the system controller is configured to automatically identify a catheter type of the catheter and adjust the operating parameters of the energy source based on the catheter type.

In various embodiments, the catheter system further comprises an inflatable balloon, including a balloon wall that defines a balloon interior, the inflatable balloon being configured to selectively receive a catheter fluid into the balloon interior.

In some embodiments, the system controller is configured to monitor and adjust balloon dimensions of the inflatable balloon.

In certain embodiments, the catheter system further comprises a plurality of emitters that is each configured to generate a plasma in the catheter fluid within the balloon interior.

In various embodiments, the system controller is configured to identify and verify an emitter operational status and an emitter configuration of each of the plurality of emitters, including determining a number of functional emitters.

In some embodiments, each of the plurality of emitters is configured to fire one or more shots of energy within the balloon interior.

In certain embodiments, the system controller is configured to monitor, track, and display a number of shots fired by each emitter of the plurality of emitters.

In various embodiments, the system controller is configured to monitor and track procedure timing data, the procedure timing data including one of: (i) an elapsed time from a beginning of a therapy cycle, and (ii) an occlusal time defined by a time period where the blood vessel is occluded by the inflatable balloon.

In some embodiments, the system controller is configured to monitor and track cycle tracking data for the catheter, the cycle tracking data including a number of cycles remaining for the catheter.

In certain embodiments, the system controller is configured to monitor and track conditions inside the inflatable balloon and the catheter such as pressure and change in pressure over time.

In various embodiments, the catheter system further comprises a graphical user interface that is communicatively coupled to the system controller, the graphical user interface being configured to (i) display catheter system information including at least one of energy source information and energy guide information, and (ii) cooperate with the system controller to control operation of the energy source.

In some embodiments, the graphical user interface is configured to display catheter information about the catheter.

In certain embodiments, the catheter information includes the type of catheter being used.

In various embodiments, the graphical user interface is configured to display procedure timing data.

In some embodiments, the procedure timing data includes one of (i) an elapsed time from a beginning of a therapy cycle, and (ii) an occlusal time defined by a time period where the blood vessel is occluded by the inflatable balloon.

In certain embodiments, the graphical user interface is configured to display cycle tracking data about the catheter.

In various embodiments, the cycle tracking data includes a number of cycles remaining for the catheter.

In some embodiments, the graphical user interface is configured to display pressure inside the inflatable balloon and change in pressure over the course of a procedure.

The present invention is also directed toward a catheter system usable for treating a treatment site within or adjacent to a vessel wall of a blood vessel or a heart valve within a body of a patient. The catheter system includes an energy source that generates energy, a catheter, an inflatable balloon, a plurality of emitters and a system controller. The system controller is coupled to the energy source. The catheter includes an energy guide that receives the energy from the energy source. The inflatable balloon includes a balloon wall that defines a balloon interior, the inflatable balloon being configured to selectively receive a catheter fluid into the balloon interior. The plurality of emitters is each configured to generate a plasma in the catheter fluid within the balloon interior. The system controller is configured to (i) monitor real-time operating parameters of the catheter system, including at least energy delivery characteristics and treatment site conditions, (ii) detect and identify operating parameters that require adjustment, and (iii) automatically adjust operating parameters.

In certain embodiments, the catheter system further includes a communicator that is coupled to the system controller, the communicator being configured to communicate treatment feedback and system status updates.

In various embodiments, the system controller is configured to automatically identify a catheter type of the catheter and adjust operating parameters of the energy source based on the catheter type.

In some embodiments, the system controller is configured to identify and verify an emitter operational status and an emitter configuration of each of the plurality of emitters, including determining a number of functional emitters.

In certain embodiments, each of the plurality of emitters is configured to fire one or more shots of energy within the balloon interior.

In various embodiments, the system controller is configured to monitor, track, and display a number of shots fired by each emitter of the plurality of emitters.

In some embodiments, the system controller is configured to monitor and track procedure timing data, the procedure timing data including one of: (i) an elapsed time from a beginning of a therapy cycle, and (ii) an occlusal time defined by a time period where the blood vessel is occluded by the inflatable balloon.

In certain embodiments, the system controller is configured to monitor and track cycle tracking data for the catheter, the cycle tracking data including a number of cycles remaining for the catheter.

The present invention is also directed toward a catheter system usable for treating a treatment site within or adjacent to a vessel wall of a blood vessel or a heart valve within a body of a patient. The catheter system includes an energy source that generates energy, a catheter, an inflatable balloon, a plurality of emitters and a system controller. The system controller is coupled to the energy source. The catheter includes an energy guide that receives the energy from the energy source. The inflatable balloon includes a balloon wall that defines a balloon interior, the inflatable balloon being configured to selectively receive a catheter fluid into the balloon interior. The plurality of emitters is each configured to generate a plasma in the catheter fluid within the balloon interior. The system controller is configured to (i) monitor real-time operating parameters of the catheter system, including at least energy delivery characteristics and treatment site conditions, (ii) detect and identify operating parameters that require adjustment, (iii) automatically adjust operating parameters, (iv) a number of shots fired by each emitter of the plurality of emitters and (v) dynamically provide a summary of system status, treatment progress, and actionable alerts.

The present invention is also directed toward a catheter system usable for treating a treatment site within or adjacent to a vessel wall of a blood vessel or a heart valve within a body of a patient. The catheter system includes an energy source, a catheter, a system controller, and a graphical user interface. The energy source generates energy. The catheter includes an energy guide that receives the energy from the energy source. The system controller is coupled to the energy source. The graphical user interface is communicatively coupled to the system controller, the graphical user interface being configured to (i) display catheter system information including at least one of energy source information and energy guide information, and (ii) cooperate with the system controller to control operation of the energy source.

The present invention is also directed toward a catheter system usable for treating a treatment site within or adjacent to a vessel wall of a blood vessel or a heart valve within a body of a patient. The catheter system includes an energy source, an energy guide, a catheter, a plurality of emitters, a system controller, and a graphical user interface. The energy source generates energy. The energy guide receives the energy from the energy source. The catheter is coupled to the energy guide. The catheter includes an inflatable balloon including a balloon wall that defines a balloon interior. The plurality of emitters is configured to generate a plasma in a catheter fluid within the balloon interior. The system controller is coupled to the energy source, the system controller being configured to: (i) monitor real-time operating parameters of the catheter system, including at least energy delivery characteristics and treatment site conditions, (ii) detect and identify operating parameters that require adjustment, (iii) automatically adjust operating parameters, (iv) a number of shots fired by each emitter of the plurality of emitters and (v) dynamically provide a summary of system status, treatment progress, and actionable alerts. The graphical user interface is communicatively coupled to the system controller, the graphical user interface being configured to display at least one of (i) catheter system information including energy source information, (ii) energy guide information, (iii) catheter information, (iv) balloon dimension information, (v) a number of the plurality of emitters, (vi) a number of shots fired by each of the plurality of emitters, the graphic user interface being configured to cooperate with the system controller to control operation of one of the energy source, the catheter, and the inflatable balloon.

In various embodiments, the graphical user interface includes an emitter control that is configured to (i) individually select each emitter in the plurality of emitters, and (ii) individually activate each emitter in the plurality of emitters.

In some embodiments, the emitter control is configured to provide a visual indicator of a number of emitters in the plurality of emitters.

In certain embodiments, the emitter control includes a touchscreen control for each emitter in the plurality of emitters.

This summary is an overview of some of the teachings of the present Application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified schematic cross-sectional view illustration of an embodiment of a catheter system in accordance with various embodiments, the catheter system including a system controller and a graphical user interface having features of the present invention;

FIG. 2A is a simplified illustration of one embodiment of the graphical user interface of the catheter system, the graphical user interface displaying representative data at a first time (t₁);

FIG. 2B is another simplified illustration of the graphical user interface illustrated in FIG. 2A, the graphical user interface displaying representative data at a second time (t₂) that is different than the first time (t₁);

FIG. 2C is another simplified illustration of the graphical user interface illustrated in FIG. 2A, the graphical user interface displaying representative data at a third time (t₃) that is different than each of the first time (t₁) and the second time (t₂);

FIG. 2D is another simplified illustration of the graphical user interface illustrated in FIG. 2A, the graphical user interface displaying representative data at a fourth time (t₄) that is different than each of the first, second, and third times (t₁₋₃);

FIG. 2E is another simplified illustration of the graphical user interface illustrated in FIG. 2A, the graphical user interface displaying representative data at a fifth time (t₅) that is different than each of the first, second, third, and fourth times (t₁₋₄);

FIG. 2F is another simplified illustration of the graphical user interface illustrated in FIG. 2A, the graphical user interface displaying representative data at a sixth time (t₆) that is different than each of the first, second, third, fourth, and fifth times (t₁₋₅); and

FIG. 3 is another embodiment of the graphical user interface illustrating a graph showing pressure changes over time during a therapy cycle.

While embodiments of the present invention are susceptible to various modifications and alternative forms, specifics thereof have been shown by way of examples and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Treatment of vascular lesions can reduce major adverse events or death in affected subjects. As referred to herein, a major adverse event is one that can occur anywhere within the body due to the presence of a vascular lesion. Major adverse events can include, but are not limited to, major adverse cardiac events, major adverse events in the peripheral or central vasculature, major adverse events in the brain, major adverse events in the musculature, or major adverse events in any of the internal organs.

In various embodiments, the catheter systems and related methods disclosed herein can include a catheter configured to advance to a vascular lesion, such as a calcified vascular lesion or a fibrous vascular lesion, at a treatment site located within or adjacent to a vessel wall of a blood vessel or a heart valve within a body of a patient. As used herein, the terms “treatment site,” “intravascular lesion,” and “vascular lesion” are used interchangeably unless otherwise noted. As such, the intravascular lesions and/or the vascular lesions are sometimes referred to herein as “lesions.”

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention, as illustrated in the accompanying drawings. The same or similar nomenclature and/or reference indicators will be used throughout the drawings, and the following detailed description to refer to the same or like parts.

In the interest of clarity, not all of the routine features of the implementations described herein are shown and described. It is appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is recognized that such a development effort might be complex and time-consuming, but would nevertheless be a routine undertaking of engineering for those of ordinary skill in the art having the benefit of this disclosure.

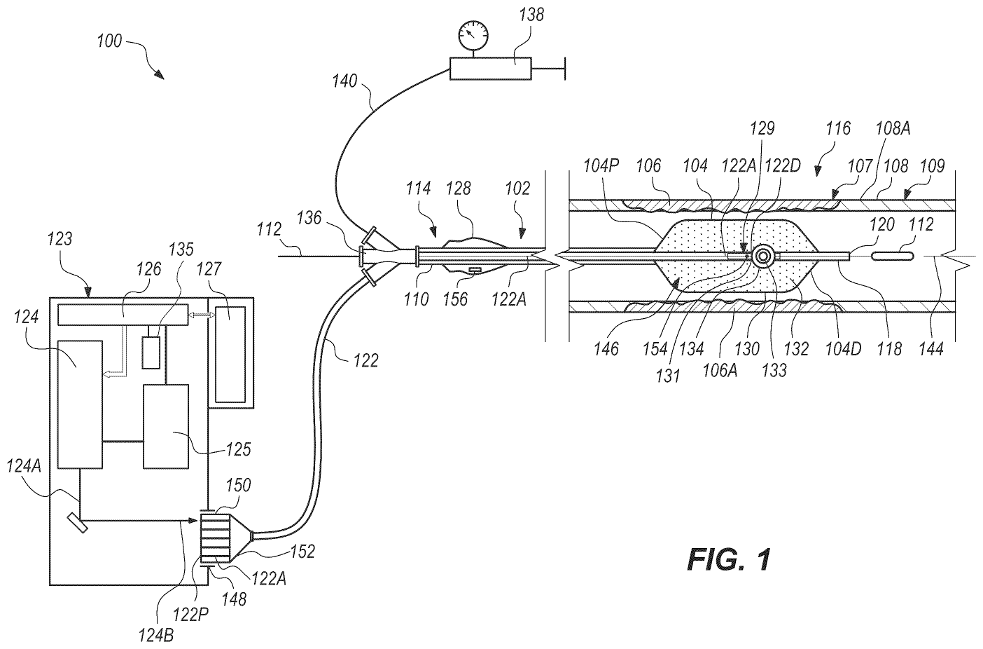

The catheter systems disclosed herein can include many different forms. Referring now to FIG. 1, a simplified schematic cross-sectional view illustration is shown of a catheter system 100 in accordance with various embodiments. The catheter system 100 is suitable for imparting pressure waves to induce fractures at one or more treatment sites within or adjacent to a vessel wall of a blood vessel or adjacent to a heart valve within a body of a patient. In the embodiment illustrated in FIG. 1, the catheter system 100 can include one or more of a catheter 102 including an energy guide bundle 122 including one or more energy guides 122A, a handle assembly 128, and an emitter assembly 129, a source manifold 136, a fluid pump 138, and a system console 123 including one or more of an energy source 124, a power source 125, a system controller 126, and a graphic user interface 127 (a “GUI”). In various embodiments, the emitter assembly 129 includes and/or incorporates at least one emitter 131 that is configured to direct and/or concentrate energy toward one or more vascular lesions 106A at a treatment site 106 within or adjacent to a vessel wall 108A of a blood vessel 108 or a heart valve within a body 107 of a patient 109. Alternatively, the catheter system 100 can include more components or fewer components than those specifically illustrated and described in relation to FIG. 1.

The catheter 102 is configured to move to the treatment site 106 within or adjacent to the vessel wall 108A of the blood vessel 108 or a heart valve within the body 107 of the patient 109. The treatment site 106 can include one or more vascular lesions 106A such as calcified vascular lesions, for example. Additionally, or in the alternative, the treatment site 106 can include vascular lesions 106A, such as fibrous vascular lesions. Still, alternatively, in some implementations, the catheter 102 can be used at a treatment site 106 within or adjacent to a heart valve within the body 107 of the patient 109.

The catheter 102 can include an inflatable balloon 104 (sometimes referred to herein simply as a “balloon”), a catheter shaft 110, and a guidewire 112. The balloon 104 can be coupled to the catheter shaft 110. The balloon 104 can include a balloon proximal end 104P and a balloon distal end 104D. The catheter shaft 110 can extend from a proximal portion 114 of the catheter system 100 to a distal portion 116 of the catheter system 100. The catheter shaft 110 can include a longitudinal axis 144. The catheter 102 and/or the catheter shaft 110 can also include a guidewire lumen 118, which is configured to move over the guidewire 112. As utilized herein, the guidewire lumen 118 defines a conduit through which the guidewire 112 extends. The catheter shaft 110 can further include an inflation lumen (not shown) and/or various other lumens for various other purposes. In some embodiments, the catheter 102 can have a distal end opening 120 and can accommodate and be tracked over the guidewire 112 as the catheter 102 is moved and positioned at or near the treatment site 106. In some embodiments, the balloon proximal end 104P can be coupled to the catheter shaft 110, and the balloon distal end 104D can be coupled to the guidewire lumen 118. The catheter 102 can include all of the components shown in FIG. 1 that are distal to the guide proximal end 122P, and the system console 123 can include all of the components shown in FIG. 1 that are proximal to the guide proximal end 122P.

The balloon 104 includes a balloon wall 130 that defines a balloon interior 146. The balloon 104 can be selectively inflated with a catheter fluid 132 to expand from a deflated state suitable for advancing the catheter 102 through a patient's vasculature, to an inflated state (as shown in FIG. 1) suitable for anchoring the catheter 102 in position relative to the treatment site 106. Stated in another manner, when the balloon 104 is in the inflated state, the balloon wall 130 of the balloon 104 is configured to be positioned substantially adjacent to the treatment site 106. It is appreciated that although FIG. 1 illustrates the balloon wall 130 of the balloon 104 being shown spaced apart from the treatment site 106 of the blood vessel 108 or a heart valve when in the inflated state, this is done for ease of illustration. It is recognized that the balloon wall 130 of the balloon 104 will typically be substantially directly adjacent to and/or abutting the treatment site 106 when the balloon 104 is in the inflated state.

As described, in various embodiments, the catheter system 100 and/or the emitter assembly 129 can include the at least one emitter 131 that is configured to transmit energy from the energy source 124 into the balloon interior 146 in order to generate plasma and/or pressure waves in the catheter fluid 132 within the balloon interior 146. Each of the emitters 131 includes a guide distal end 122D of one of the energy guides 122A, which is positioned within the balloon interior 146, and a corresponding plasma target 133 (also sometimes referred to as a “plasma generating structure” or a “plasma generator”) that is positioned near, but typically spaced apart from, the guide distal end 122D. As referred to herein, the plasma target 133 or “plasma generator” can include and/or incorporate any suitable type of structure that is located at or near the guide distal end 122D of the energy guide 122A. Energy from the energy source 124 is directed toward and received by the energy guide 122A, is guided through the energy guide 122A, and is then emitted from the guide distal end 122D of the energy guide 122A. The energy emitted from the guide distal end 122D is directed toward and impinges on and energizes the corresponding plasma target 133 for purposes of generating the plasma in the catheter fluid 132 within the balloon interior 146.

In many embodiments, the present invention utilizes a laser light source or other suitable light source as the energy source 124, and is configured to shine laser light energy onto the plasma target 133 to cause plasma generation via interaction with a plasma target material rather than optical breakdown of the catheter fluid 132. This moves the plasma creation away from the guide distal end 122D of the energy guide 122A (which can be an optical fiber in some embodiments). This can be accomplished by positioning the plasma target 133 away from the guide distal end 122D of the energy guide 122A to absorb the light energy and convert it into a plasma at some distance away from the guide distal end 122D of the energy guide 122A.

The balloon 104 suitable for use in the catheter system 100 includes those that can be passed through the vasculature of a patient 109 when in the deflated state. In some embodiments, the balloons 104 are made from silicone. In other embodiments, the balloon 104 can be made from materials such as polydimethylsiloxane (PDMS), polyurethane, polymers such as PEBAX™ material, nylon, or any other suitable material.

The balloon 104 can have any suitable diameter (in the inflated state). In various embodiments, the balloon 104 can have a diameter (in the inflated state) ranging from less than one millimeter (mm) up to 25 mm. In some embodiments, the balloon 104 can have a diameter (in the inflated state) ranging from at least 1.5 mm up to 14 mm. In some embodiments, the balloon 104 can have a diameter (in the inflated state) ranging from at least two mm up to five mm.

In some embodiments, the balloon 104 can have a length ranging from at least three mm to 300 mm. More particularly, in some embodiments, the balloon 104 can have a length ranging from at least eight mm to 200 mm. It is appreciated that a balloon 104 having a relatively longer length can be positioned adjacent to larger treatment sites 106, and, thus, may be usable for imparting pressure waves onto and inducing fractures in larger vascular lesions 106A or multiple vascular lesions 106A at precise locations within the treatment site 106. It is further appreciated that a longer balloon 104 can also be positioned adjacent to multiple treatment sites 106 at any one given time.

The balloon 104 can be inflated to inflation pressures of between approximately one atmosphere (atm) and 70 atm. In some embodiments, the balloon 104 can be inflated to inflation pressures of from at least 20 atm to 60 atm. In other embodiments, the balloon 104 can be inflated to inflation pressures of from at least six atm to 20 atm. In still other embodiments, the balloon 104 can be inflated to inflation pressures of from at least three atm to 20 atm. In yet other embodiments, the balloon 104 can be inflated to inflation pressures of from at least two atm to ten atm.

The balloon 104 can have various shapes, including, but not to be limited to, a conical shape, a square shape, a rectangular shape, a spherical shape, a conical/square shape, a conical/spherical shape, an extended spherical shape, an oval shape, a tapered shape, a bone shape, a stepped diameter shape, an offset shape, or a conical offset shape. In some embodiments, the balloon 104 can include a drug-eluting coating or a drug-eluting stent structure. The drug-eluting coating or drug-eluting stent can include one or more therapeutic agents, including anti-inflammatory agents, anti-neoplastic agents, anti-angiogenic agents, and the like.

The catheter fluid 132 can be a liquid or a gas. Some examples of the catheter fluid 132 suitable for use can include, but are not limited to one or more of water, saline, contrast medium, fluorocarbons, perfluorocarbons, gases, such as carbon dioxide, or any other suitable catheter fluid 132. In some embodiments, the catheter fluid 132 can be used as a base inflation fluid. In some embodiments, the catheter fluid 132 can include a mixture of saline to contrast medium in a volume ratio of approximately 50:50. In other embodiments, the catheter fluid 132 can include a mixture of saline to contrast medium in a volume ratio of approximately 25:75. In still other embodiments, the catheter fluid 132 can include a mixture of saline to contrast medium in a volume ratio of approximately 75:25. However, it is understood that any suitable ratio of saline to contrast medium can be used. The catheter fluid 132 can be tailored on the basis of composition, viscosity, and the like so that the rate of travel of the pressure waves are appropriately manipulated. In certain embodiments, the catheter fluids 132 suitable for use are biocompatible. A volume of catheter fluid 132 can be tailored by the chosen energy source 124 and the type of catheter fluid 132 used.

In certain embodiments, the catheter fluid 132 can include a wetting agent or surface-active agent (surfactant). These compounds can lower the tension between solid and liquid matter. These compounds can act as emulsifiers, dispersants, detergents, and water infiltration agents. Wetting agents or surfactants reduce surface tension of the liquid and allow it to fully wet and come into contact with optical components (such as the energy guide(s) 122A) and mechanical components (such as other portions of the emitter assembly 129). This reduces or eliminates the accumulation of bubbles and pockets or inclusions of gas within the emitter assembly 129. Nonexclusive examples of chemicals that can be used as wetting agents include, but are not limited to, Benzalkonium Chloride, Benzethonium Chloride, Cetylpyridinium Chloride, Poloxamer 188, Poloxamer 407, Polysorbate 20, Polysorbate 40, and the like. Non-exclusive examples of surfactants can include, but are not limited to, ionic and non-ionic detergents, and Sodium stearate. Another suitable surfactant is 4-(5-dodecyl) benzenesulfonate. Other examples can include docusate (dioctyl sodium sulfosuccinate), alkyl ether phosphates, and perfluorooctanesulfonate (PFOS), to name a few.

The catheter fluids 132 can include those that include absorptive agents that can selectively absorb light in the ultraviolet region (e.g., at least ten nanometers (nm) to 400 nm), the visible region (e.g., at least 400 nm to 780 nm), or the near-infrared region (e.g., at least 780 nm to 2.5 μm) of the electromagnetic spectrum. Suitable absorptive agents can include those with absorption maxima along the spectrum from at least ten nm to 2.5 μm. Alternatively, the catheter fluids 132 can include those that include absorptive agents that can selectively absorb light in the mid-infrared region (e.g., at least 2.5 μm to 15 μm), or the far-infrared region (e.g., at least 15 μm to one mm) of the electromagnetic spectrum. In various embodiments, the absorptive agent can be those that have an absorption maximum matched with the emission maximum of the laser used in the catheter system 100. By way of non-limiting examples, various lasers usable in the catheter system 100 can include neodymium:yttrium-aluminum-garnet (Nd:YAG−emission maximum=1064 nm) lasers, holmium:YAG (Ho:YAG−emission maximum=2.1 μm) lasers, or erbium:YAG (Er:YAG−emission maximum=2.94 μm) lasers. In some embodiments, the absorptive agents can be water-soluble. In other embodiments, the absorptive agents are not water-soluble. In some embodiments, the absorptive agents used in the catheter fluids 132 can be tailored to match the peak emission of the energy source 124. Various energy sources 124 having emission wavelengths of at least ten nanometers to one millimeter are discussed elsewhere herein.

The catheter shaft 110 of the catheter 102 can be coupled to the one or more energy guides 122A of the energy guide bundle 122 that are in optical communication with the energy source 124. The energy guide(s) 122A can be disposed along the catheter shaft 110 and within the balloon 104. In some embodiments, each energy guide 122A can be an optical fiber, and the energy source 124 can be a laser. The energy source 124 can be in optical communication with the energy guides 122A at the proximal portion 114 of the catheter system 100.

In some embodiments, the catheter shaft 110 can be coupled to multiple energy guides 122A, such as a first energy guide, a second energy guide, a third energy guide, etc., which can be disposed at any suitable positions about and/or relative to the guidewire lumen 118 and/or the catheter shaft 110. For example, in certain non-exclusive embodiments, two energy guides 122A can be spaced apart from one another by approximately 180 degrees about the circumference of the guidewire lumen 118 and/or the catheter shaft 110; three energy guides 122A can be spaced apart from one another by approximately 120 degrees about the circumference of the guidewire lumen 118 and/or the catheter shaft 110; four energy guides 122A can be spaced apart from one another by approximately 90 degrees about the circumference of the guidewire lumen 118 and/or the catheter shaft 110; or six energy guides 122A can be spaced apart from one another by approximately 60 degrees about the circumference of the guidewire lumen 118 and/or the catheter shaft 110. Still alternatively, multiple energy guides 122A need not be uniformly spaced apart from one another about the circumference of the guidewire lumen 118 and/or the catheter shaft 110. More particularly, it is further appreciated that the energy guides 122A can be disposed uniformly or non-uniformly about the guidewire lumen 118 and/or the catheter shaft 110 to achieve the desired effect in the desired locations.

In certain embodiments, the guidewire lumen 118 can have a grooved outer surface, with the grooves extending in a generally longitudinal direction along the guidewire lumen 118. In such embodiments, each of the energy guides 122A and/or the emitter(s) 131 of the emitter assembly 129 can be positioned, received, and retained within an individual groove formed along and/or into the outer surface of the guidewire lumen 118. Alternatively, the guidewire lumen 118 can be formed without a grooved outer surface, and the position of the energy guides 122A and/or the emitter(s) 131 of the emitter assembly 129 relative to the guidewire lumen 118 can be maintained in another suitable manner.

The catheter system 100 and/or the energy guide bundle 122 can include any number of energy guides 122A in optical communication with the energy source 124 at the proximal portion 114, and with the catheter fluid 132 within the balloon interior 146 of the balloon 104 at the distal portion 116. For example, in some embodiments, the catheter system 100 and/or the energy guide bundle 122 can include from one energy guide 122A to greater than 30 energy guides 122A. Alternatively, in other embodiments, the catheter system 100 and/or the energy guide bundle 122 can include greater than 30 energy guides 122A.

The energy guides 122A can have any suitable design for the purposes of generating plasma and/or pressure waves in the catheter fluid 132 within the balloon interior 146. Thus, the general description of the energy guides 122A as light guides is not intended to be limiting in any manner, except for as set forth in the claims appended hereto. More particularly, although the catheter systems 100 are often described with the energy source 124 as a light source, and the one or more energy guides 122A as light guides, the catheter system 100 can alternatively include any suitable energy source 124 and energy guides 122A for purposes of generating the desired plasma in the catheter fluid 132 within the balloon interior 146. For example, in one non-exclusive alternative embodiment, the energy source 124 can be configured to provide high-voltage electrical pulses, and each energy guide 122A can include an electrode pair including spaced apart electrodes that extend into the balloon interior 146. In such embodiment, each pulse of high voltage is applied to the electrodes and forms an electrical arc across the electrodes, which, in turn, generates the plasma and forms the pressure waves in the catheter fluid 132 that are utilized to provide the fracture force onto the vascular lesions 106A at the treatment site 106. Still, alternatively, the energy source 124 and/or the energy guides 122A can have another suitable design and/or configuration.

In certain embodiments, the energy guides 122A can include an optical fiber or flexible light pipe. The energy guides 122A can be thin and flexible and can allow light signals to be sent with very little loss of strength. The energy guides 122A can include a core surrounded by a cladding about its circumference. In some embodiments, the core can be a cylindrical core or a partially cylindrical core. The core and cladding of the energy guides 122A can be formed from one or more materials, including but not limited to one or more types of glass, silica, or one or more polymers. The energy guides 122A may also include a protective coating, such as a polymer. It is appreciated that the index of refraction of the core will be greater than the index of refraction of the cladding.

Each energy guide 122A can guide energy along its length from a guide proximal end 122P to the guide distal end 122D having at least one optical window (not shown) that is positioned within the balloon interior 146.

The energy guides 122A can assume many configurations about and/or relative to the catheter shaft 110 of the catheter 102. In some embodiments, the energy guides 122A can run parallel to the longitudinal axis 144 of the catheter shaft 110. In some embodiments, the energy guides 122A can be physically coupled to the catheter shaft 110. In other embodiments, the energy guides 122A can be disposed along a length of an outer diameter of the catheter shaft 110. In yet other embodiments, the energy guides 122A can be disposed within one or more energy guide lumens within the catheter shaft 110.

The energy guides 122A can also be disposed at any suitable positions about the circumference of the guidewire lumen 118 and/or the catheter shaft 110, and the guide distal end 122D of each of the energy guides 122A can be disposed at any suitable longitudinal position relative to the length of the balloon 104 and/or relative to the length of the guidewire lumen 118 to more effectively and precisely impart pressure waves for purposes of disrupting the vascular lesions 106A at the treatment site 106.

In certain embodiments, the energy guides 122A can include one or more photoacoustic transducers 154, where each photoacoustic transducer 154 can be in optical communication with the energy guide 122A within which it is disposed. In some embodiments, the photoacoustic transducers 154 can be in optical communication with the guide distal end 122D of the energy guide 122A. In such embodiments, the photoacoustic transducers 154 can have a shape that corresponds with and/or conforms to the guide distal end 122D of the energy guide 122A.

The photoacoustic transducer 154 is configured to convert light energy into an acoustic wave at or near the guide distal end 122D of the energy guide 122A. The direction of the acoustic wave can be tailored by changing an angle of the guide distal end 122D of the energy guide 122A.

In certain embodiments, the photoacoustic transducers 154 disposed at the guide distal end 122D of the energy guide 122A can assume the same shape as the guide distal end 122D of the energy guide 122A. For example, in certain non-exclusive embodiments, the photoacoustic transducer 154 and/or the guide distal end 122D can have a conical shape, a convex shape, a concave shape, a bulbous shape, a square shape, a stepped shape, a half-circle shape, an ovoid shape, and the like. The energy guide 122A can further include additional photoacoustic transducers 154 disposed along one or more side surfaces of the length of the energy guide 122A.

In some embodiments, the energy guides 122A and/or the emitter assembly 129 can further include one or more diverting structures or “diverters” (not shown in FIG. 1), such as within the energy guide 122A and/or near the guide distal end 122D of the energy guide 122A, that are configured to direct energy from the energy guide 122A toward a side surface which can be located at or near the guide distal end 122D of the energy guide 122A, before the energy is directed toward the balloon wall 130. A diverting structure can include any structure of the system that diverts energy from the energy guide 122A away from its axial path toward a side surface of the energy guide 122A. The energy guides 122A can each include one or more optical windows disposed along the longitudinal or circumferential surfaces of each energy guide 122A and in optical communication with a diverting structure. Stated in another manner, the diverting structures can be configured to direct energy in the energy guide 122A toward a side surface that is at or near the guide distal end 122D, where the side surface is in optical communication with an optical window. The optical windows can include a portion of the energy guide 122A that allows energy to exit the energy guide 122A from within the energy guide 122A, such as a portion of the energy guide 122A lacking a cladding material on or about the energy guide 122A.

Examples of the diverting structures suitable for use include a reflecting element, a refracting element, and a fiber diffuser. The diverting structures suitable for focusing energy away from the guide distal end 122D of the energy guide 122A can include, but are not to be limited to, those having a convex surface, a gradient-index (GRIN) lens, and a mirror focus lens. Upon contact with the diverting structure, the energy is diverted within the energy guide 122A to one or more of the plasma target 133 and the photoacoustic transducer 154 that is in optical communication with a side surface of the energy guide 122A. When utilized, the plasma target 133 receives energy emitted from the guide distal end 122D of the energy guide 122A to generate plasma in the catheter fluid 132 within the balloon interior 146, which, in turn, causes the creation of plasma bubbles and/or pressure waves that can be directed away from the side surface of the energy guide 122A and toward the balloon wall 130. Additionally, or in the alternative, when utilized, the photoacoustic transducer 154 converts light energy into an acoustic wave that extends away from the side surface of the energy guide 122A.

Additionally, or in the alternative, in certain embodiments, such diverting structures that can be incorporated into the energy guides 122A, can also be incorporated into the design of the emitter assembly 129 and/or the plasma target 133 for purposes of directing and/or concentrating acoustic and mechanical energy toward specific areas of the balloon wall 130 in contact with the vascular lesions 106A at the treatment site 106 to impart pressure onto and induce fractures in such vascular lesions 106A.

The source manifold 136 can be positioned at or near the proximal portion 114 of the catheter system 100. The source manifold 136 can include one or more proximal end openings that can receive the one or more energy guides 122A of the energy guide bundle 122, the guidewire 112, and/or an inflation conduit 140 that is coupled in fluid communication with the fluid pump 138. The catheter system 100 can also include the fluid pump 138 that is configured to inflate the balloon 104 with the catheter fluid 132 as needed.

As noted above, in the embodiment illustrated in FIG. 1, the system console 123 includes one or more of the energy source 124, the power source 125, the system controller 126, and the GUI 127. Alternatively, the system console 123 can include more components or fewer components than those specifically illustrated in FIG. 1. For example, in certain non-exclusive alternative embodiments, the system console 123 can be designed without the GUI 127. Still alternatively, one or more of the energy source 124, the power source 125, the system controller 126, and the GUI 127 can be provided within the catheter system 100 without the specific need for the system console 123.

As shown, the system console 123, and the components included therewith, is operatively coupled to the catheter 102, the energy guide bundle 122, and the remainder of the catheter system 100. For example, in some embodiments, as illustrated in FIG. 1, the system console 123 can include a console connection aperture 148 (also sometimes referred to generally as a “socket”) by which the energy guide bundle 122 is mechanically coupled to the system console 123. In such embodiments, the energy guide bundle 122 can include a guide coupling housing 150 (also sometimes referred to generally as a “ferrule”) that houses a portion, such as the guide proximal end 122P, of each of the energy guides 122A. The guide coupling housing 150 is configured to fit and be selectively retained within the console connection aperture 148 to provide the mechanical coupling between the energy guide bundle 122 and the system console 123.

The energy guide bundle 122 can also include a guide bundler 152 (or “shell”) that brings each of the individual energy guides 122A closer together so that the energy guides 122A and/or the energy guide bundle 122 can be in a more compact form as it extends with the catheter 102 into the blood vessel 108 or the heart valve during use of the catheter system 100.

The energy source 124 can be selectively and/or alternatively coupled in optical communication with each of the energy guides 122A, such as to the guide proximal end 122P of each of the energy guides 122A, in the energy guide bundle 122. In particular, the energy source 124 is configured to generate energy in the form of a source beam 124A, such as a pulsed source beam, that can be selectively and/or alternatively directed to and received by each of the energy guides 122A in the energy guide bundle 122, such as through the use of a multiplexer (not shown), as an individual guide beam 124B. Alternatively, the catheter system 100 can include more than one energy source 124. For example, in one non-exclusive alternative embodiment, the catheter system 100 can include a separate energy source 124 for each of the energy guides 122A in the energy guide bundle 122.

The energy source 124 can have any suitable design. In certain embodiments, the energy source 124 can be configured to provide sub-millisecond pulses of energy from the energy source 124 that are focused onto a small spot in order to couple it into the guide proximal end 122P of the energy guide 122A. Such pulses of energy are then directed and/or guided along the energy guides 122A to a location within the balloon interior 146 of the balloon 104, thereby inducing plasma formation in the catheter fluid 132 within the balloon interior 146 of the balloon 104, such as via the plasma target 133 that can be located at or near the guide distal end 122D of the energy guide 122A. In particular, in such embodiments, the energy emitted at the guide distal end 122D of the energy guide 122A is directed toward and energizes the plasma target 133 to form the plasma in the catheter fluid 132 within the balloon interior 146. The plasma formation can initiate a pressure wave and can initiate the rapid formation of one or more bubbles that can rapidly expand to a maximum size and then dissipate through a cavitation event that can also launch a pressure wave upon collapse. An exemplary plasma-induced bubble 134 is illustrated in FIG. 1. The rapid expansion of the plasma-induced bubbles 134 can generate one or more pressure waves within the catheter fluid 132 and thereby impart pressure waves upon the treatment site 106. The pressure waves can transfer mechanical energy through an incompressible catheter fluid 132 to the treatment site 106 to impart a fracture force on the vascular lesions 106A at the treatment site 106. Without wishing to be bound by any particular theory, it is believed that the rapid change in catheter fluid 132 momentum upon the balloon wall 130 of the balloon 104 that is in contact with or positioned near the vascular lesions 106A at the treatment site 106 is transferred to the vascular lesions 106A to induce fractures in the vascular lesions 106A.

In various non-exclusive alternative embodiments, the sub-millisecond pulses of energy from the energy source 124 can be delivered to the treatment site 106 at a frequency of between approximately one hertz (Hz) and 5000 Hz, between approximately 30 Hz and 1000 Hz, between approximately ten Hz and 100 Hz, or between approximately one Hz and 30 Hz. Alternatively, the sub-millisecond pulses of energy can be delivered to the treatment site 106 at a frequency that can be greater than 5000 Hz or less than one Hz, or any other suitable range of frequencies.

It is appreciated that although the energy source 124 is typically utilized to provide pulses of energy, the energy source 124 can still be described as providing a single source beam 124A, i.e., a single pulsed source beam.

The energy sources 124 suitable for use can include various types of light sources including lasers and lamps. Alternatively, the energy sources 124 can include any suitable type of energy source.

Suitable lasers can include short pulse lasers on the sub-millisecond timescale. In some embodiments, the energy source 124 can include lasers on the nanosecond (ns) timescale. The lasers can also include short pulse lasers on the picosecond (ps), femtosecond (fs), and microsecond (μs) timescales. It is appreciated that there are many combinations of laser wavelengths, pulse widths, and energy levels that can be employed to achieve plasma in the catheter fluid 132 of the catheter 102. In various non-exclusive alternative embodiments, the pulse widths can include those falling within a range including from at least ten ns to 3000 ns, at least 20 ns to 100 ns, or at least one ns to 500 ns. Alternatively, any other suitable pulse width range can be used.

Exemplary nanosecond lasers can include those within the UV to IR spectrum, spanning wavelengths of about ten nanometers (nm) to one millimeter (mm). In some embodiments, the energy sources 124 suitable for use in the catheter systems 100 can include those capable of producing light at wavelengths of from at least 750 nm to 2000 nm. In other embodiments, the energy sources 124 can include those capable of producing light at wavelengths of from at least 700 nm to 3000 nm. In still other embodiments, the energy sources 124 can include those capable of producing light at wavelengths of from at least 100 nm to ten micrometers (μm). Nanosecond lasers can include those having repetition rates of up to 200 KHz.

In some embodiments, the laser can include a Q-switched thulium:yttrium-aluminum-garnet (Tm:YAG) laser. In other embodiments, the laser can include a neodymium:yttrium-aluminum-garnet (Nd:YAG) laser, holmium:yttrium-aluminum-garnet (Ho:YAG) laser, erbium:yttrium-aluminum-garnet (Er:YAG) laser, excimer laser, helium-neon laser, carbon dioxide laser, as well as doped, pulsed, fiber lasers.

In still other embodiments, the energy source 124 can include a plurality of lasers that are grouped together in series. In yet other embodiments, the energy source 124 can include one or more low energy lasers that are fed into a high energy amplifier, such as a master oscillator power amplifier (MOPA). In still yet other embodiments, the energy source 124 can include a plurality of lasers that can be combined in parallel or in series to provide the energy needed to create the plasma bubble 134 in the catheter fluid 132.

The catheter system 100 can generate pressure waves having maximum pressures in the range of at least one megapascal (MPa) to 100 MPa. The maximum pressure generated by a particular catheter system 100 will depend on the energy source 124, the absorbing material, the bubble expansion, the propagation medium, the balloon material, and other factors. In various non-exclusive alternative embodiments, the catheter systems 100 can generate pressure waves having maximum pressures in the range of at least approximately two MPa to 50 MPa, at least approximately two MPa to 30 MPa, or approximately at least 15 MPa to 25 MPa.

The pressure waves can be imparted upon the treatment site 106 from a distance within a range from at least approximately 0.1 millimeters (mm) to greater than approximately 25 mm extending radially from the energy guides 122A when the catheter 102 is placed at the treatment site 106. In various non-exclusive alternative embodiments, the pressure waves can be imparted upon the treatment site 106 from a distance within a range from at least approximately ten mm to 20 mm, at least approximately one mm to ten mm, at least approximately 1.5 mm to four mm, or at least approximately 0.1 mm to ten mm extending radially from the energy guides 122A when the catheter 102 is placed at the treatment site 106. In other embodiments, the pressure waves can be imparted upon the treatment site 106 from another suitable distance that is different than the foregoing ranges. In some embodiments, the pressure waves can be imparted upon the treatment site 106 within a range of at least approximately two MPa to 30 MPa at a distance from at least approximately 0.1 mm to ten mm. In some embodiments, the pressure waves can be imparted upon the treatment site 106 from a range of at least approximately two MPa to 25 MPa at a distance from at least approximately 0.1 mm to ten mm. Still alternatively, other suitable pressure ranges and distances can be used.

The power source 125 is electrically coupled to and is configured to provide necessary power to each of the energy source 124, the system controller 126, the GUI 127, and the handle assembly 128. The power source 125 can have any suitable design for such purposes.

The system controller 126 can be electrically coupled to and receives power from the power source 125. The system controller 126 can be coupled to and is configured to control the operation of each of the energy source 124 and the GUI 127. The system controller 126 can include one or more processors or circuits for purposes of controlling the operation of at least the energy source 124 and the GUI 127. For example, the system controller 126 can control the energy source 124 for generating pulses of energy as desired and/or at any desired firing rate.

The system controller 126 can also be configured to control the operation of other components of the catheter system 100, such as the positioning of the catheter 102 adjacent to the treatment site 106, the inflation of the balloon 104 (including the control of pressure inside the balloon 104) with the catheter fluid 132, etc. In some embodiments, the system controller 126 can include an automatic endoflator that can be used to monitor and/or adjust pressure levels inside of the balloon 104.

Further, or in the alternative, the catheter system 100 can include one or more additional controllers that can be positioned in any suitable manner for purposes of controlling the various operations of the catheter system 100. For example, in certain embodiments, an additional controller and/or a portion of the system controller 126 can be positioned and/or incorporated within the handle assembly 128. The system controller 126 can query the energy source 124 to determine its operating status and conditions, control the pulse shape and energy directly, and set the pulse rate.

The system controller 126 can monitor the real-time operating parameters of the energy source 124. The operating parameters of the catheter system 100 can include energy delivery characteristics of the energy source 124 and conditions of the treatment site 106, as non-limiting, non-exclusive examples. The system controller 126 can process the output pulse characteristic data of the energy source 124. The system controller 126 can monitor pulse energy, pulse shape, and/or pulse envelope parameters of the energy source 124 using calibrated photodiode-based detectors and FPGA circuitry included in the system controller 126.

The system controller 126 can analyze data collected from the catheter 102 to adjust the operating parameters of the energy source 124 to meet target parameters. The pulse envelope shape of the energy source 124 can be controlled by the system controller 126 directly by driving current to one or more fiber amplifier stages. The energy source 124 can have a set of values for pulse energy, pulse envelope parameters (for example, pulse rise time, pulse width, top third percent, etc.), repetition rate for each catheter 102 type, and treatment indication. The system controller 126 can improve the operation of the energy source 124 by attempting to maintain these values near their targets by dynamic adjustment upon deviation.

The system controller 126 can also monitor an energy source temperature of the energy source 124. In some embodiments, the energy source 124 can include a seed laser. Because the pulse shape of the energy source 124 can vary with the temperature of the seed laser components, monitoring and adjusting the temperature levels can lead to improved performance of the energy source 124 during treatment. In some embodiments, the system controller can use heating and cooling to adjust the seed laser operating parameters to control pulse shape.

The system controller 126 can interface directly with the energy source 124 to monitor an energy source status of the energy source 124 and control the energy source 124 through predefined commands and user-defined sets of commands. For example, in one non-limiting, non-exclusive embodiment, a command sets a drive current on a fiber preamplifier in the seed laser stage, controlling the pulse shape as it varies with energy source 124 temperature. These commands, that can be executable by the system controller 126, are automatic so that the user does not need to be involved in any processes other than treatment, simplifying the treatment process for the user.

The system controller 126 can identify when the operating parameters require adjustment. Upon identification, the system controller 126 can automatically adjust energy delivery parameters to improve lithotripsy treatment efficacy and safety based on the identified operating parameters. Based on the stage of treatment, the system controller 126 can dynamically provide a prioritized summary of the system status, treatment progress, and actionable alerts to the user based on the monitored operating parameters.

The operating parameters for the catheter 102, such as pulse energy, envelope shape, repetition rate, number of emitter stations, etc., can be stored in the system memory of the system controller 126. The system controller 126 can control the system console 123 and the energy source 124 to provide the target pulse characteristics and energize the correct energy guide 122A channels without user intervention.

The system controller 126 can be configured to automatically identify the type of catheter 102 that is used in the catheter system 100. Upon identification of the catheter type, the system controller 126 can adjust the operating parameters of the energy guides 122A and the energy source 124 based on the identified catheter type to improve performance and safety.

The system controller 126 can be configured to monitor the balloon dimensions of the inflatable balloon 104. Based on sensor feedback, the system controller 126 can adjust the balloon dimensions of the inflatable balloon in real-time to improve treatment conditions at the treatment site 106.

The system controller 126 can be configured to monitor balloon pressure of the inflatable balloon 104. For example, the system controller 126 can monitor changes in the balloon pressure of the inflatable balloon 104 as the treatment procedure progresses. The system controller 126 can automatically adjust the balloon pressure in order to improve the efficacy of the treatment procedure.

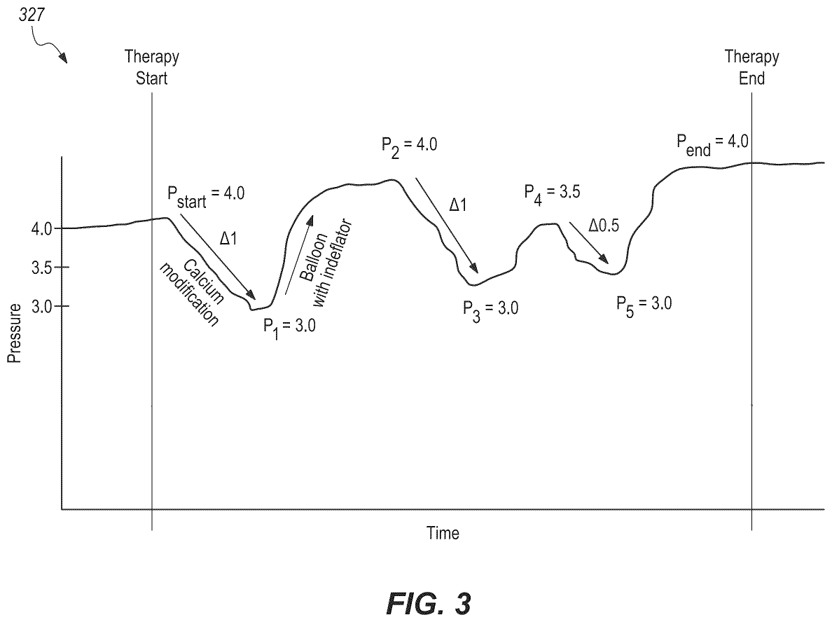

The system controller 126 can be configured to monitor and track procedure timing data during a treatment cycle, such as illustrated in FIG. 3. Among other data, the procedure timing data can include (i) an elapsed time from a beginning of a therapy cycle, and (ii) an occlusal time defined by a time period where the blood vessel 108 is occluded by the inflatable balloon 104. This information can be provided to the user via the GUI 127.

The GUI 127 is accessible by the user or operator of the catheter system 100. The GUI 127 is communicatively coupled to the system controller 126. With such a design, the GUI 127 can be used by the user or operator to ensure that the catheter system 100 is effectively utilized to impart pressure onto and induce fractures into the vascular lesions 106A at the treatment site 106. The GUI 127 can provide the user or operator with information that can be used before, during, and after the use of the catheter system 100. In one embodiment, the GUI 127 can provide static visual data and/or information to the user or operator. In addition, or in the alternative, the GUI 127 can provide dynamic visual data and/or information to the user or operator, such as video data or any other data that changes over time during the use of the catheter system 100. In various embodiments, the GUI 127 can include one or more colors, different sizes, varying brightness, etc., that may act as alerts to the user or operator. Additionally, or in the alternative, the GUI 127 can provide audio data, haptic feedback, or other information to the user or operator. The GUI 127 can also provide the user or operator with control of other components of the catheter system 100. The specifics of the GUI 127 can vary depending upon the design requirements of the catheter system 100, or the specific needs, specifications, and/or desires of the user or operator.

As shown in FIG. 1, the handle assembly 128 can be positioned at or near the proximal portion 114 of the catheter system 100, and/or near the source manifold 136. In this embodiment, the handle assembly 128 is coupled to the balloon 104 and is positioned spaced apart from the balloon 104. Alternatively, the handle assembly 128 can be positioned at another suitable location.

The handle assembly 128 is handled and used by the user or operator to operate, position, and control the catheter 102. The design and specific features of the handle assembly 128 can vary to suit the design requirements of the catheter system 100. In the embodiment illustrated in FIG. 1, the handle assembly 128 is separate from, but in electrical and/or fluid communication with one or more of the system controller 126, the energy source 124, the fluid pump 138, and the GUI 127. In some embodiments, the handle assembly 128 can integrate and/or include at least a portion of the system controller 126 within an interior of the handle assembly 128. For example, as shown, in certain such embodiments, the handle assembly 128 can include circuitry 156 that can form at least a portion of the system controller 126. In one embodiment, the circuitry 156 can include a printed circuit board having one or more integrated circuits, or any other suitable circuitry. In an alternative embodiment, the circuitry 156 can be omitted, or can be included within the system controller 126, which in various embodiments can be positioned outside of the handle assembly 128, such as within the system console 123. It is understood that the handle assembly 128 can include fewer or additional components than those specifically illustrated and described herein.

In various embodiments, as noted above, the emitter assembly 129 includes and/or incorporates the at least one emitter 131 that is configured to transmit energy from the energy source 124 into the balloon interior 146 so that plasma and/or pressure waves are generated in the catheter fluid 132 within the balloon interior. Each emitter 131 includes the guide distal end 122D of one of the energy guides 122A and the corresponding plasma target 133 that is positioned near, but typically spaced apart from, the guide distal end 122D. Additionally, in many embodiments, each emitter 131 further includes an emitter housing 260 that is configured to maintain the desired positioning between the guide distal end 122D of the energy guide 122A and the plasma target 133, and to direct and/or concentrate energy generated in the catheter fluid 132 within the balloon interior 146 so as to impart pressure onto and induce fractures in vascular lesions 106A at the treatment site 106.

During the use of the catheter system 100, the energy guide 122A receives the energy from the energy source 124 and guides the energy from the guide proximal end 122P to the guide distal end 122D. The energy is then emitted from the guide distal end 122D of the energy guide 122A so that the energy is directed toward and impinges on and energizes the corresponding plasma target 133 for purposes of generating the plasma in the catheter fluid 132 within the balloon interior 146. The plasma generation then forms the pressure waves in the catheter fluid 132 that are directed toward the vascular lesions 106A at the treatment site 106 to provide the fracture force onto the vascular lesions 106A at the treatment site 106.

The plasma target 133 can be formed from any suitable material that is configured to generate the desired plasma in the catheter fluid 132 within the balloon interior 146 when the energy is directed from the guide distal end 122D of the energy guide 122A to impinge on the plasma target 133.

The catheter system 100 can also include a communicator 135 that is coupled to the system controller 126. The communicator 135 can be configured to provide treatment progress, treatment feedback, and system status updates to the user. The data communicated by the communicator 135 can be provided to the user via the graphical user interface 127. The design and specific features of the communicator 135 can vary to suit the design requirements of the catheter system 100.

Various alternative embodiments of GUI 127 are illustrated and described in detail below within subsequent Figures.

As with all embodiments illustrated and described herein, various structures may be omitted from the figures for clarity and ease of understanding. Additionally, the figures may include certain structures that can be omitted without deviating from the intent and scope of the invention. It is further recognized that the structures included in the various figures shown and described herein are not necessarily drawn to scale for ease of viewing and/or understanding.

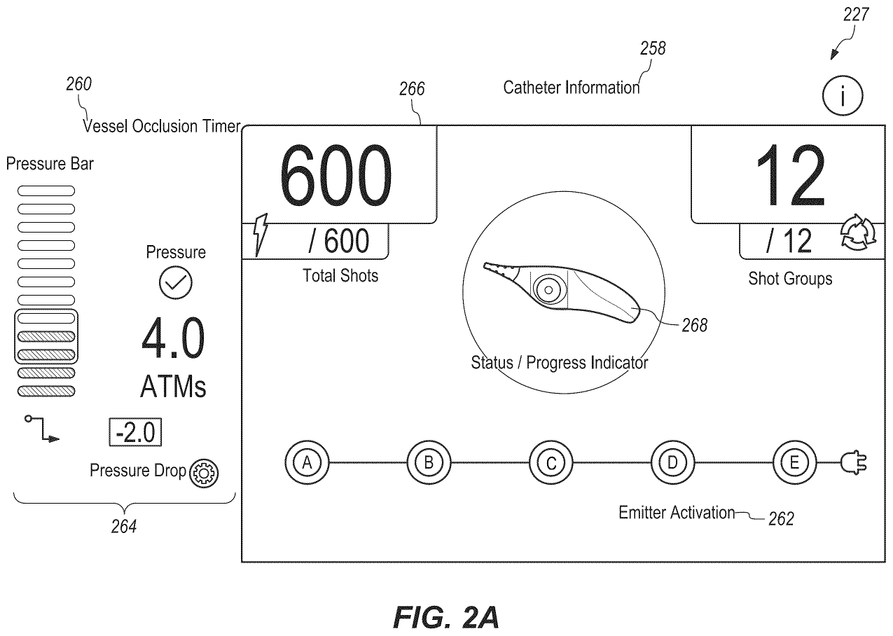

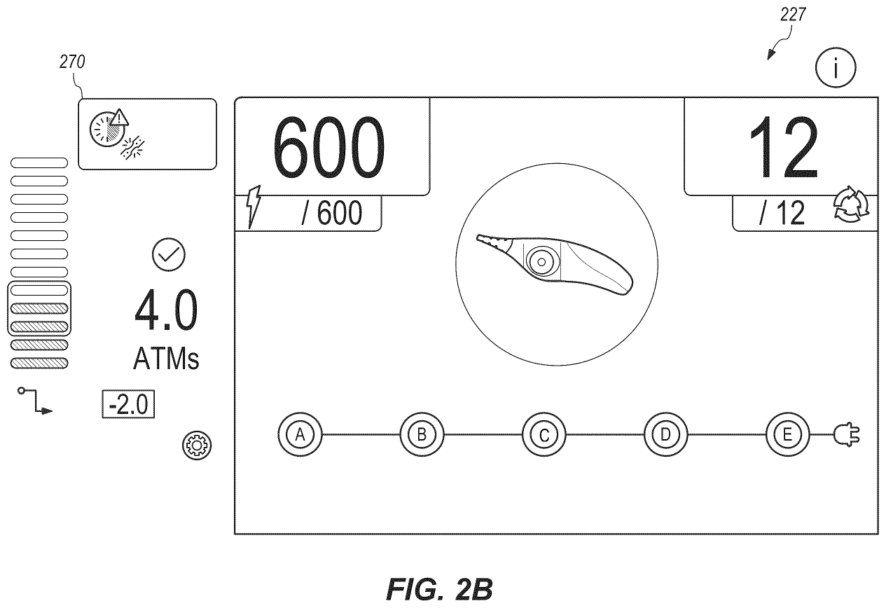

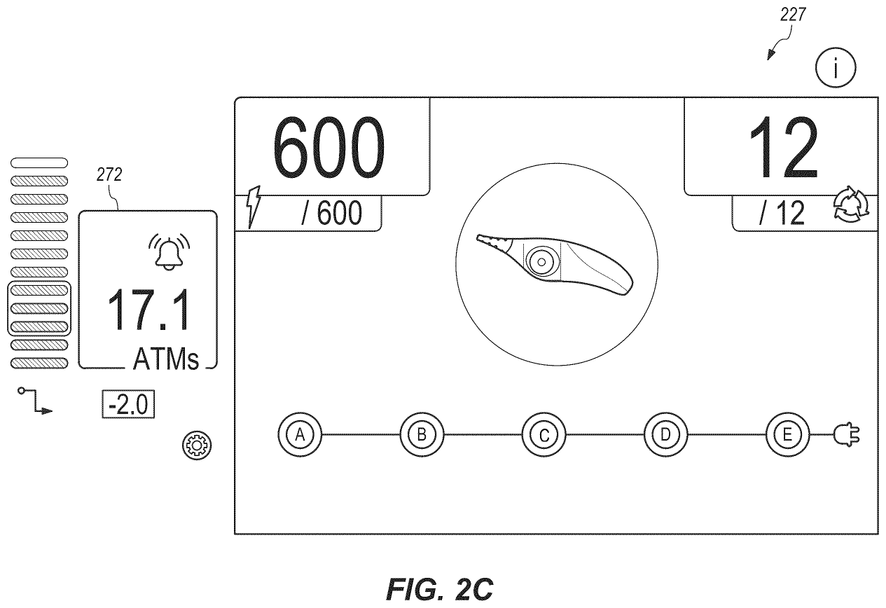

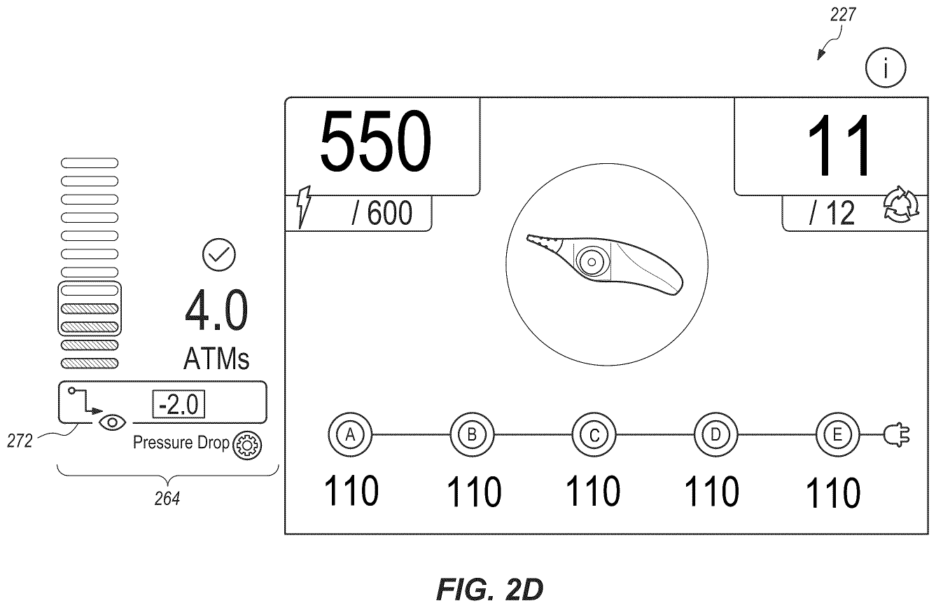

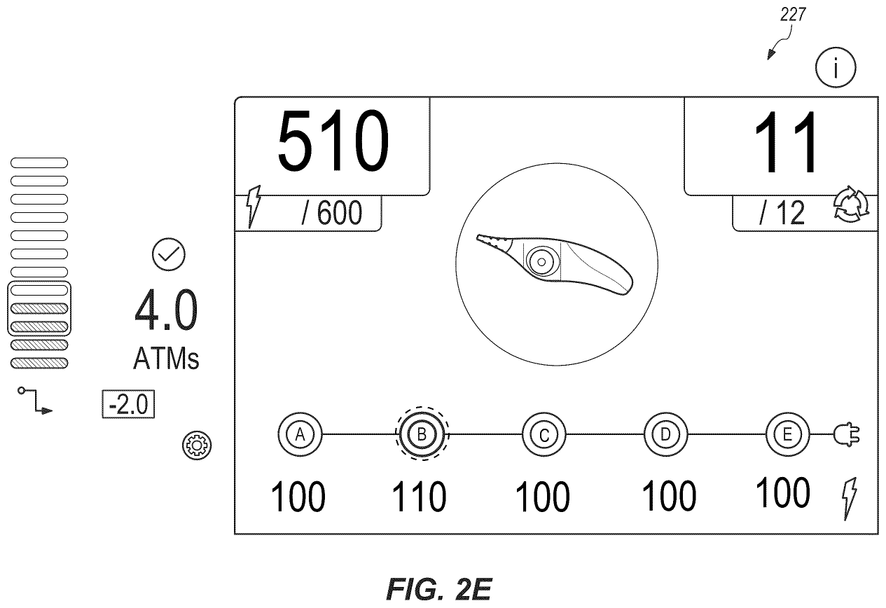

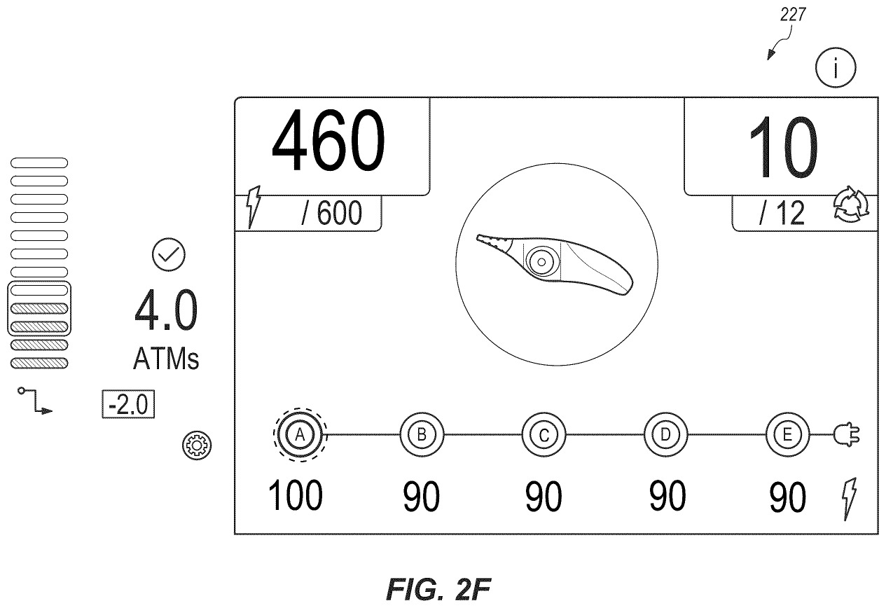

FIG. 2A is an embodiment of the graphical user interface 227 of the catheter system 100 (illustrated in FIG. 1). The graphical user interface 227 can display representative data, taken at a first time (t₁). FIG. 2A is illustrative of one possible layout of the graphical user interface 227 with the catheter 102 (illustrated in FIG. 1) inserted into the system console 123 (illustrated in FIG. 1) and activated.

In the embodiment shown in FIG. 2A, the graphical user interface 227 can include the following functional display areas: (1) catheter information 258, (2) timers 260, (3) emitter control 262, (4) pressure monitor 264, (5) shot counter 266, and (6) activation state and progress 268.

The catheter information 258 can include information about a catheter type of the catheter 102. For example, suitable catheter types can include above-the-knee, below-the-knee, or coronary artery disease catheters. The catheter information 258 can include emitter information such as the number of emitters 131 (illustrated in FIG. 1) and/or the individual status of each of the emitters 131. The catheter information 258 can include data, dimensions, and/or statistics of the balloon 104 (illustrated in FIG. 1). The catheter information 258 can also include cycle counting, including the number of cycles for the catheter 102 that is inserted into the system console 123 (illustrated in FIG. 1), as well as the total number of cycles remaining for the inserted catheter 102. The specifics of the catheter information 258 can vary depending upon the design requirements of the catheter system 100, or the specific needs, specifications, and/or desires of the user or operator.

The catheter information 258 can be displayed on the graphical user interface 227 so that information is displayed on the interface in a graphical or numerical format, making it easier for the user to monitor and understand the status of the catheter 102 and the progress of each treatment cycle. The user is not required to be involved with setting the important parameters that provide the target performance of a given catheter or any of the controls during operation. The user can set key operating parameters, for example, whether a given emitter station is on or off, balloon inflation, balloon pressure, and energize the catheter on or off for any given amount of time or number of cycles deemed necessary during treatment. The graphical user interface 227 can track all this information for the user in a convenient graphical or numeric format so that the user can focus on the treatment and progress/efficacy. The graphical user interface 227 can provide the catheter information 258 and controls available at a glance, thereby reducing the cognitive load on the user.

The timers 260 can include timing information such as: (i) elapsed time from the beginning of the procedure, and (ii) time that the blood vessel is occluded by the balloon 104, also referred to herein as the “occluded vessel timer.” The timers 260 can also include custom timers set and/or started by the user or operator. The specifics of the timers 260 can vary depending upon the design requirements of the catheter system 100, or the specific needs, specifications, and/or desires of the user or operator.

The emitter control 262 can include individual emitter 131 selection and/or activation. The emitter control 262 can provide a visual indicator of the number of emitters provided by the inserted catheter 102. The emitter control 262 can include one or more touchscreen controls for each emitter 131, allowing the user or operator to individually or collectively activate and deactivate each of the emitters 131. The system controller 126 (illustrated in FIG. 1) can be configured to identify and/or verify an emitter operational status and configuration of each of the emitters 131, including determining the number of functional emitters 131, to improve plasma generation during treatment. The specifics of the emitter control 262 can vary depending upon the design requirements of the catheter system 100, or the specific needs, specifications, and/or desires of the user or operator.

The pressure monitor 264 can include data on the pressure levels inside of the catheter 102 and/or balloon 104. The balloon 104 pressure can be displayed by the graphical user interface 227 in real-time, with indicators for desired ranges of suitable pressures for the balloon 104. The balloon 104 pressure data can be captured by a pressure sensor. In some embodiments, the pressure sensor can be located in the handle assembly 128 (illustrated in FIG. 1). The specifics of the pressure monitor 264 can vary depending upon the design requirements of the catheter system 100, or the specific needs, specifications, and/or desires of the user or operator.

The pressure monitor 264 can include a safety interlock feature that ensures the balloon 104 is inflated to a target level before the energy source 124 is activated. The pressure monitor 264 can monitor rapid pressure drops to provide indications of balloon rupturing. The pressure monitor 264 can also monitor gradual pressure changes, often in time intervals, that indicate the progress of the procedure as vessel walls become pliable, allowing the balloon 104 to expand. The pressure monitor 264 can display pressure drops over the course of a therapy cycle. The pressure monitor 264 can include a graphical display bar of the pressure, including desired pressure zones for therapy.

The shot counter 266 can include the initial number of shots for the catheter 102 that is currently inserted into the system console 123. The shot counter 266 can also include the total number of shots fired and the total number of shots remaining for the inserted catheter 102. The shot counter 266 can include an indication of the number of shots fired for each individual emitter 131 location within the catheter 102. The specifics of the shot counter 266 can vary depending upon the design requirements of the catheter system 100, or the specific needs, specifications, and/or desires of the user or operator.

The activation state and progress 268 can display the status of the catheter 102 and its availability for activation. The activation state and progress 268 can provide information on the activation of the catheter 102 as well as overall treatment progress. The specifics of the activation state and progress 268 can vary depending upon the design requirements of the catheter system 100, or the specific needs, specifications, and/or desires of the user or operator.

FIG. 2B is the graphical user interface 227 illustrated in FIG. 2A taken at a second time (t₂) that is different than the first time (t₁). The graphical user interface 227 displays representative data at the second time (t₂). FIG. 2B is illustrative of one possible layout of the graphical user interface 227 with the catheter 102 (illustrated in FIG. 1) inserted into the system console 123 (illustrated in FIG. 1) and activated.

While the first and second times (t₁₋₂) are referred to as “first” and “second” times, it is understood that these times could represent the graphical user interface 227 display data that is taken at any time that is different than another time. For example, the second time (t₂) could occur before or after the first time (t₁). It is understood that the “first,” “second,” “third,” etc. times (t_{1-X}) described herein could have any sequential order and the use of these identifiers is for purposes of identification and differentiation only and is not intended to be relevant with respect to temporal sequencing.

The embodiment illustrated in FIG. 2B includes an occluded vessel timer 270. The occluded vessel timer 270 can be activated when the inflatable balloon 104 (illustrated in FIG. 1) is inflated past a set pressure limit and obstructs or impedes blood flow in the blood vessel 108A (illustrated in FIG. 1).

The occluded vessel timer 270 of the graphical user interface 227 can show the inflation pressure on the stacked bar graph in digital format. For example, at the time (t₂) illustrated in FIG. 2B, the desired balloon pressure is set at 4 ATM. In certain embodiments, the readout and bar of the occluded vessel timer 270 can turn green to indicate the proximity to the desired balloon pressure. The safety interlock feature that uses catheter pressure is disabled once the pressure is raised above a set threshold, such as 1 ATM in one non-exclusive, non-limiting embodiment. In some non-limiting, non-exclusive embodiments, the pressure readout and activation button in the handle assembly 128 (illustrated in FIG. 1) and on the graphical user interface 227 are turned from blue to green (or any suitable change in color), indicating that the catheter 102 is ready to fire. The specifics of the occluded vessel timer 270 can vary depending upon the design requirements of the catheter system 100, or the specific needs, specifications, and/or desires of the user or operator.

FIG. 2C is the graphical user interface 227 illustrated in FIG. 2A taken at a third time (t₃) that is different than each of the first time (t₁) and the second time (t₂). The graphical user interface 227 displays representative data at the third time (t₃). FIG. 2C is illustrative of one possible layout of the graphical user interface 227 with the catheter 102 (illustrated in FIG. 1) inserted into the system console 123 (illustrated in FIG. 1) and activated.

The embodiment illustrated in FIG. 2C includes an alert 272 for pressure over a target pressure range. In some non-limiting, non-exclusive embodiments, the readout and bar graph turn yellow (or any suitable color) to indicate this condition. The specifics of the alert 272 can vary depending upon the design requirements of the catheter system 100, or the specific needs, specifications, and/or desires of the user or operator.

FIG. 2D is the graphical user interface 227 illustrated in FIG. 2A taken at a fourth time (t₄) that is different than each of the first, second, and third times (t₁₋₃). The graphical user interface 227 displays representative data taken at the fourth time (t₄). FIG. 2D is illustrative of one possible layout of the graphical user interface 227 with the catheter 102 (illustrated in FIG. 1) inserted into the system console 123 (illustrated in FIG. 1) and activated.

The embodiment illustrated in FIG. 2D includes one non-exclusive embodiment of an indicator for the pressure drop monitor, which is an alert 272 set by the user. For example, the pressure drop monitor can vary in color to indicate various changes in pressure. This feature can alert the user when the balloon 104 (illustrated in FIG. 1) pressure drops by a specified amount set by the user (at the fourth time (t₄) illustrated by FIG. 2D, a 2 ATM drop was measured over a therapy cycle, one example of the therapy cycle is illustrated in FIG. 3), providing an indication that the vascular lesion 106A (illustrated in FIG. 1) in the vessel wall 108A (illustrated in FIG. 1) has been fractured allowing the blood vessel 108 to expand. This information can be displayed in the pressure monitoring 264 display location.

Alternatively, suppose the user does not want to turn the alert ‘on.’ In that case, the graphical user interface 227 can display the pressure drop at the end of the therapy cycle without an alert notification. Additionally, the graphical user interface 227 can be set to terminate the therapy cycle if a user-specified pressure drop threshold is achieved.

FIG. 2E is the graphical user interface 227 illustrated in FIG. 2A taken at a fifth time (t₅) that is different than each of the first, second, third, and fourth times (t₁₋₄). The graphical user interface 227 displays representative data taken at the fifth time (t₅). FIG. 2E is illustrative of one possible layout of the graphical user interface 227 with the catheter 102 (illustrated in FIG. 1) inserted into the system console 123 (illustrated in FIG. 1) and activated.

The embodiment illustrated in FIG. 2E can include an emitter control 262. The emitter control 262 can be activated through a touchscreen of the graphical user interface 227 by pressing an icon (shown as emitters A-E) for the desired emitter. Using the emitter control 262, the user can select and/or deselect any number of emitters as desired to turn such emitters on/off. In this case, the second to last emitter distally, which is identified as a B, has been turned off. The deselection/off function of emitter B is illustrated in FIG. 2E as a circle shown in dashed lines, while the selection/on function of emitters A and C-D is shown with standard circles.

It is appreciated that the on/off and/or selection/deselection functions of emitter selector can be illustrated/played/provided on the graphical user interface 227 using any suitable visual (e.g., the use of varying colors, shapes, shading, graphics, etc.), audio, and/or haptic feedback cues that are provided to the user. The total number of shots available for each emitter can be indicated below the icon (for purposes of illustration, is shown as 120 in FIG. 2E). In some embodiments, the number of shots available can decrease as the procedure progresses. Emitters that are turned off and are not fired will stay constant (e.g., will remain at 120).

FIG. 2F is the graphical user interface 227 illustrated in FIG. 2A taken at a sixth time (t₆) that is different than each of the first, second, third, fourth, and fifth times (t₁₋₅). The graphical user interface 227 displays representative data taken at the sixth time (t₆). FIG. 2F is illustrative of one possible layout of the graphical user interface 227 with the catheter 102 (illustrated in FIG. 1) inserted into the system console 123 (illustrated in FIG. 1) and activated.

The embodiment illustrated in FIG. 2F can include an example of shots fired countdown for emitters. In FIG. 2F, Emitter A has been turned off. The other four emitters (B-E) have run through two (2) cycles including varying numbers of shots. The cycle counter has been decreased by two (2), and the total shot count decreased by one hundred forty (140), as illustrated on the graphical user interface 227 shown in FIG. 2F. Also shown in FIG. 2F, Emitters B through D have been decreased by varying numbers of shots and Emitter A still has one hundred (100) shots available.

FIG. 3 is another embodiment of the graphical user interface 327, illustrating a graph showing pressure changes over time during a therapy cycle. As used herein, the “therapy cycle” is understood to mean the time while the treatment activation is active. In other words, as shown in FIG. 3, the therapy cycle is the time from the “Therapy Start” to the “Therapy End.”

Therapy Start can indicate when the treatment activation starts. For example, the treatment activation can start when a user of the catheter system 100 (illustrated in FIG. 1) engages a button on a handle assembly 128 (illustrated in FIG. 1).

Therapy End can indicate when the treatment activation ends. For example, the treatment activation can end when a user of the catheter system 100 releases the button on the handle assembly 128. In other embodiments, the treatment activation can end when the maximum number of energy pulses is reached.

The pressure monitor 264 (shown in FIGS. 2A-2F) of the graphical user interface 227 (shown in FIG. 2A-2F) can illustrate the change in pressure (e.g., as shown in FIG. 3, P₁/P₂/P₃/P₄/P₅-Pₛₜₐᵣₜ) at any point during the therapy cycle. At the Therapy End, the pressure monitor 264 can illustrate the change in pressure from Therapy Start to Therapy End (e.g., as shown in FIG. 3, P_{end}-Pₛₜₐᵣₜ) as a summary until the next treatment activation. In the example shown in FIG. 2F, this summary is shown as −2.0 ATM. In contrast, the therapy cycle shown in FIG. 3 would result in no change in pressure, e.g., 0 ATM.

In certain embodiments, the pressure monitor 264 can accumulate all of the pressure changes across the therapy cycle. For example, assuming the pressure change from Pₛₜₐᵣₜ to P₁ is −1 ATM but is −2.5 ATM at P_{end}. In other words, a 1 ATM drop from the start to P₁, a drop of 1 ATM from P₂ to P₃, and a final drop of 0.5 ATM from P₃ to P₄. By displaying these pressure changes in each stage (e.g., P₂ to P₃), a decrease in pressure in the inflatable balloon 104 (illustrated in FIG. 1) during a treatment activation indicates that calcium is being modified. Conversely, an increase in pressure in the inflatable balloon 104 indicates the user is applying more pressure to the balloon 104 to compensate for the drop in pressure. All of this information can be displayed in the pressure monitor 264 on the graphical user interface 127.

The present technology is also directed toward methods for treating a treatment site within or adjacent to a vessel wall, with such methods utilizing the devices disclosed herein.

In summary, based on the various embodiments of the present invention illustrated and described in detail herein, the catheter systems and related methods can include a catheter configured to advance to a vascular lesion, such as a calcified vascular lesion, or a fibrous vascular lesion, at a treatment site located within or adjacent a blood vessel within a body of a patient. The catheter includes a catheter shaft, and an inflatable balloon that is coupled and/or secured to the catheter shaft. The balloon can include a balloon wall that defines a balloon interior. The balloon can be configured to receive a catheter fluid within the balloon interior to expand from a deflated state suitable for advancing the catheter through a patient's vasculature, to an inflated state suitable for anchoring the catheter in position relative to the treatment site.

In certain embodiments, the catheter systems and related methods utilize an energy source, e.g., a light source such as a laser source or another suitable energy source, which provides energy that is guided by one or more energy guides, e.g., light guides such as optical fibers, which are disposed along the catheter shaft and within the balloon interior of the balloon to create a localized plasma in the catheter fluid that is retained within the balloon interior of the balloon. The energy guide can be used in conjunction with a plasma generator that is positioned at or near a guide distal end of the energy guide within the balloon interior of the balloon located at the treatment site. The creation of the localized plasma can initiate a pressure wave and can initiate the rapid formation of one or more bubbles that can rapidly expand to a maximum size and then dissipate through a cavitation event that can launch a pressure wave upon collapse. The rapid expansion of the plasma-induced bubbles (also sometimes referred to simply as “plasma bubbles”) can generate one or more pressure waves in the catheter fluid retained within the balloon interior of the balloon and thereby impart pressure waves onto and induce fractures in the vascular lesions at the treatment site within or adjacent to the blood vessel wall within the body of the patient. In some embodiments, the energy source can be configured to provide sub-millisecond pulses of energy, e.g., light energy, to initiate the plasma formation in the catheter fluid within the balloon to cause the rapid bubble formation and to impart the pressure waves upon the balloon wall at the treatment site. Thus, the pressure waves can transfer mechanical energy through an incompressible catheter fluid to the treatment site to impart a fracture force on the intravascular lesion. Without wishing to be bound by any particular theory, it is believed that the rapid change in catheter fluid momentum upon the balloon wall that is in contact with the intravascular lesion is transferred to the intravascular lesion to induce fractures to the lesion.

It should be noted that, as used in this specification and the appended claims, the singular forms “a,” “an,” and “the” include plural referents unless the content and/or context clearly dictates otherwise. It should also be noted that the term “or” is generally employed in its sense, including “and/or” unless the content or context clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

It is recognized that the figures shown and described are not necessarily drawn to scale, and that they are provided for ease of reference and understanding, and for relative positioning of the structures.

The headings used herein are provided for consistency with suggestions under 37 CFR 1.77 or otherwise to provide organizational cues. These headings shall not be viewed to limit or characterize the invention(s) set out in any claims that may be issued from this disclosure. As an example, a description of a technology in the “Background” is not an admission that technology is prior art to any invention(s) in this disclosure. Neither is the “Summary” or “Abstract” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

It is understood that although a number of different embodiments of the catheter systems have been illustrated and described herein, one or more features of any one embodiment can be combined with one or more features of one or more of the other embodiments, provided that such combination satisfies the intent of the present invention.

While a number of exemplary aspects and embodiments of the catheter systems have been discussed above, those of skill in the art will recognize certain modifications, permutations, additions, and sub-combinations thereof. It is, therefore, intended that the following appended claims and claims hereafter introduced are interpreted to include all such modifications, permutations, additions, and sub-combinations as are within their true spirit and scope, and no limitations are intended to the details of construction or design herein shown.

What is claimed is:

1. A catheter system for treating a treatment site within or adjacent to a vessel wall of a blood vessel or a heart valve within a body of a patient, the catheter system comprising:
an energy source that generates energy;
a catheter that includes an energy guide that receives the energy from the energy source;
a system controller that is coupled to the energy source, the system controller being configured to: (i) monitor real-time operating parameters of the catheter system, including at least energy delivery characteristics and treatment site conditions, (ii) detect and identify operating parameters that require adjustment, (iii) automatically adjust operating parameters, and (iv) dynamically provide a summary of system status, treatment progress, and actionable alerts.

2. The catheter system of claim 1 further comprising a communicator that is coupled to the system controller, the communicator being configured to communicate treatment feedback and system status updates.

3. The catheter system of claim 1 wherein the system controller is configured to automatically identify a catheter type of the catheter and adjust operating parameters of the energy source based on the catheter type.

4. The catheter system of claim 1 further comprising an inflatable balloon including a balloon wall that defines a balloon interior, the inflatable balloon being configured to selectively receive a catheter fluid into the balloon interior.

5. The catheter system of claim 4 wherein the system controller is configured to monitor and adjust balloon dimensions of the inflatable balloon.

6. The catheter system of claim 4 further comprising a plurality of emitters that is each configured to generate a plasma in the catheter fluid within the balloon interior.

7. The catheter system of claim 6 wherein the system controller is configured to identify and verify an emitter operational status and an emitter configuration of each of the plurality of emitters, including determining a number of functional emitters.

8. The catheter system of claim 6 wherein each of the plurality of emitters is configured to fire one or more shots of energy within the balloon interior.

9. The catheter system of claim 8 wherein the system controller is configured to monitor, track, and display a number of shots fired by each emitter of the plurality of emitters.

10. The catheter system of claim 4 wherein the system controller is configured to monitor and track procedure timing data, the procedure timing data including one of: (i) an elapsed time from a beginning of a therapy cycle, and (ii) an occlusal time defined by a time period where the blood vessel is occluded by the inflatable balloon.

11. The catheter system of claim 1 wherein the system controller is configured to monitor and track cycle tracking data for the catheter, the cycle tracking data including a number of cycles remaining for the catheter.

12. A catheter system for treating a treatment site within or adjacent to a vessel wall of a blood vessel or a heart valve within a body of a patient, the catheter system comprising:
an energy source that generates energy;
a catheter that includes an energy guide that receives the energy from the energy source;
an inflatable balloon including a balloon wall that defines a balloon interior, the inflatable balloon being configured to selectively receive a catheter fluid into the balloon interior;
a plurality of emitters that is each configured to generate a plasma in the catheter fluid within the balloon interior; and
a system controller that is coupled to the energy source, the system controller being configured to: (i) monitor real-time operating parameters of the catheter system, including at least energy delivery characteristics and treatment site conditions, (ii) detect and identify operating parameters that require adjustment, and (iii) automatically adjust operating parameters.

13. The catheter system of claim 12 further comprising a communicator that is coupled to the system controller, the communicator being configured to communicate treatment feedback and system status updates.

14. The catheter system of claim 12 wherein the system controller is configured to automatically identify a catheter type of the catheter and adjust operating parameters of the energy source based on the catheter type.

15. The catheter system of claim 12 wherein the system controller is configured to identify and verify an emitter operational status and an emitter configuration of each of the plurality of emitters, including determining a number of functional emitters.

16. The catheter system of claim 12 wherein each of the plurality of emitters is configured to fire one or more shots of energy within the balloon interior.

17. The catheter system of claim 16 wherein the system controller is configured to monitor, track, and display a number of shots fired by each emitter of the plurality of emitters.

18. The catheter system of claim 12 wherein the system controller is configured to monitor and track procedure timing data, the procedure timing data including one of: (i) an elapsed time from a beginning of a therapy cycle, and (ii) an occlusal time defined by a time period where the blood vessel is occluded by the inflatable balloon.

19. The catheter system of claim 12 wherein the system controller is configured to monitor and track cycle tracking data for the catheter, the cycle tracking data including a number of cycles remaining for the catheter.

20. A catheter system for treating a treatment site within or adjacent to a vessel wall of a blood vessel or a heart valve within a body of a patient, the catheter system comprising:
an energy source that generates energy;
a catheter that includes an energy guide that receives the energy from the energy source;
an inflatable balloon including a balloon wall that defines a balloon interior, the inflatable balloon being configured to selectively receive a catheter fluid into the balloon interior;
a plurality of emitters that is each configured to generate a plasma in the catheter fluid within the balloon interior; and
a system controller that is coupled to the energy source, the system controller being configured to: (i) monitor real-time operating parameters of the catheter system, including at least energy delivery characteristics and treatment site conditions, (ii) detect and identify operating parameters that require adjustment, (iii) automatically adjust operating parameters, (iv) a number of shots fired by each emitter of the plurality of emitters and (v) dynamically provide a summary of system status, treatment progress, and actionable alerts.
